(19) United States
(12) Patent Application Publication
(10) Pub. No.: US 2018/0289656 A1
(43) Pub. Date: Oct. 11, 2018

(54) COMPOSITIONS AND METHODS TO TREAT URINARY TRACT INFECTIONS

(71) Applicant: Hennepin Life Sciences, LLC, Plymouth, MN (US)

(72) Inventors: Patrick M. Schlievert, Iowa City, IA (US); Marnie L. Peterson, Jackson, WY (US)

(21) Appl. No.: 15/765,816

(22) PCT Filed: Oct. 7, 2016
(86) PCT No.: PCT/US2016/056078

§ 371 Date: Apr. 4, 2018

Related U.S. Application Data
(60) Provisional application No. 62/241,321, filed on Oct. 14, 2015.

(51) Int. Cl. A61K 31/23; A61K 47/38; A61K 47/18; A61K 47/44; A61K 47/10; A61K 47/12; A61K 9/06; A61P 31/04

(52) U.S. Cl. CPC A61K 31/23; A61K 47/38; A61K 47/183; A61K 47/44; A61K 45/06; A61K 47/12; A61K 9/06; A61P 31/04; A61K 2300/00; A61K 47/10

(57) ABSTRACT

This invention relates to treating and preventing urinary tract infections. Glycerol monolaurate (GML) and/or GML-related compositions together with suitable accelerants in gel-based formulation may be used to treat urinary tract infections that may be generally diagnosed, for example, in women, catheterized patients and elderly individuals. Such a gel-based formulation kills, or inhibits the growth of, one or more pathogenic microorganisms that cause urinary tract infections.

### PRIORITY

This application claims the benefit of U.S. Provisional Application No. 62/241,321, entitled “COMPOSITIONS AND METHODS TO TREAT URINARY TRACT INFECTIONS” and filed Oct. 14, 2015, the entirety of which is incorporated herein by reference.

### TECHNICAL FIELD

This invention relates to treating and preventing urinary tract infections, particularly in women, catheterized patients, and elderly patients.

### SUMMARY

Glycerol monolaurate (GML) and GML-related compositions, together with suitable accelerants, in gel-based formulations may be applied to biological surfaces (skin and/or mucous membranes) to kill pathogenic microorganisms, inhibit production of exotoxins by pathogenic microorganisms, prevent inflammation and stabilize human cells to interfere with toxic reactions or infections, and select for beneficial bacteria such as lactobacilli and bifidobacteria. In particular, such gel-based formulations may be used to treat urinary tract infections that may be generally found, for example, in women, catheterized patients and elderly individuals. These urinary infections are typically caused by pathogenic microorganisms such as Escherichia coli, other Enterobacteriaceae, Staphylococcus aureus, Pseudomonas aeruginosa, and other gram positive and gram negative bacteria.

One embodiment of the present invention is a gel-based formulation comprising a composition that kills, or inhibits the growth of, one or more pathogenic microorganisms that cause urinary tract infections, where the composition comprises about 0.0001-0.05 M of an accelerant selected from the group consisting of lactic acid, ascorbic acid, citric acid, ethylenediaminetetraacetic acid (ETDA), and combinations thereof, and about 10-100 mg/mL of an active compound selected from the group consisting of Formula 1, Formula 2, and a combination of Formulas 1 and 2:

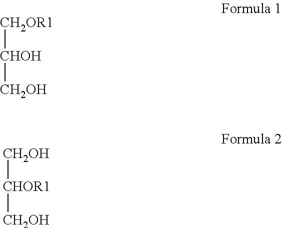

wherein R1 is: CO(CH₂)₁₀CH₃.

In an exemplary embodiment, the gel-based formulation includes GML that may be present in an amount of about 10-100 mg/mL, preferably about 30-70 mg/mL. The gel-based formulation may also include a glycol glycerol, a cellulose derivative, a plant-derived oil, and/or petroleum jelly. Still further, the gel-based formulation may include an additional active ingredient selected from an antibacterial, anti-viral, anti-fungal, anti-protozoan, or a combination thereof.

In still other embodiments, the accelerant and active compound are combined with a topical solution comprising the following components:

a) about 73.55 w/w % propylene glycol;

b) about 25 w/w % polyethylene glycol 400;

c) about 1.25 w/w % hydroxyethyl cellulose or hydroxypropyl cellulose; and

d) about 1-25 w/w % saline and/or water.

Alternatively, the accelerant and compound are combined with a topical solution comprising substantially pure or about 100% w/w % plant-derived oil, petroleum jelly or derivatives thereof.

In some these embodiments the plant-derived oil is selected from the group consisting of palm oil, olive oil, corn oil, and combinations thereof.

In other embodiments the gel-based formulation has a pH of about 4-4.5

A particular embodiment of the present invention is a gel-based formulation composition that kills, or inhibits the growth one or more pathogenic microorganisms that cause urinary tract infections, wherein the formulation comprises about 0.0001-0.05 M of an accelerant selected from the group consisting of lactic acid, ascorbic acid, citric acid, ethylenediaminetetraacetic acid, and combinations thereof, and about 10-100 mg/mL of a compound selected from the group consisting of Formula 1, Formula 2, and a combination of Formulas 1 and 2

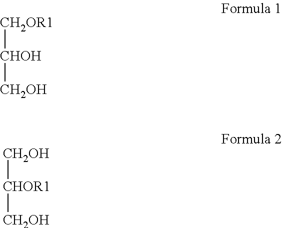

wherein R1 is: CO(CH₂)₁₀CH₃.

The accelerant and compound are combined with a topical solution comprising a) about 73.55 w/w % propylene glycol; b) about 25 w/w % polyethylene glycol 400; c) about 1.25 w/w % hydroxyethyl cellulose or hydroxypropyl cellulose; and d) about 1-25 w/w % saline and/or, water. Alternatively, the accelerant and compound are combined with a topical solution comprising a plant-derived oil; petroleum jelly, or a derivative thereof. In this embodiment, the plant-derived oil may be selected from the group consisting of palm oil, olive oil, corn oil, and combinations thereof, and the gel-based formulation has a pH of about 4-4.5.

Another embodiment of the present invention is a method of treatment or prophylaxis, the method comprising:

(a) identifying a patient having a urinary tract infection or at risk of such an infection that is or could be caused by one or more pathogenic microorganisms; and

(b) administering to the patient a gel-based formulation that (i) kills, or inhibits the growth of, the one or more pathogenic microorganisms, and (ii) comprises at least one accelerant and a compound selected from the group consisting of Formula 1, Formula 2, and a combination of Formulas 1 and 2:

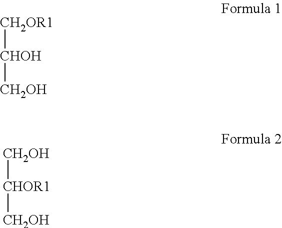

wherein R1 is: CO(CH₂)₁₀CH₃.

In this embodiment of the present invention, the accelerant may be lactic acid, ascorbic acid, citric acid, ethylenediaminetetraacetic acid, or other chelating ingredients, the compound may GML, and the gel-based formulation may include a plant-derived oil selected from the group consisting of palm oil, olive oil, corn oil, and combinations thereof, a cellulose derivative selected from the group consisting of hydroxyethyl and hydroxypropyl cellulose, a glycol derivative selected from the group consisting of polyethylene and propylene glycol, a petroleum jelly derivative, as well as water and/or saline. Such embodiments may also include an additional active ingredient selected from the group consisting of an antibacterial, an anti-viral, an anti-fungal, anti-protozoan, and combinations thereof.

Alternative embodiments of the present invention may include a gel-based formulation that contains compound (either together with or in place of Formulas 1 and 2), that are either Formula 3 or Formula 4, or both Formula 3 and Formula 4.

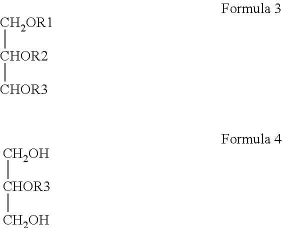

wherein R1 may be: hydrogen, CO(CH₂)₈CH₃, CO(CH₂)₁₀CH₃, or CO(CH₂)₁₂CH₃;

R2 may be: hydrogen, CO(CH₂)₈CH₃, CO(CH₂)₁₀CH₃, CO(CH₂)₁₂CH₃, O(CH₂)₉CH₃, O(CH₂)₁₁CH₃, or O(CH₂)₁₃CH₃, and

R3 may be: CO(CH₂)₈CH₃, CO(CH₂)₁₀CH₃, CO(CH₂)₁₂CH₃, O(CH₂)₉CH₃, O(CH₂)₁₁CH₃, or O(CH₂)₁₃CH₃.

The gel-based formulations of the present invention may be administered either before, simultaneous with, or after the administration of one or more supplementary ingredients. Supplementary ingredients can include, for example, anti-fungal ingredients, modulators of immune function, or antibiotics. In addition, a urinary catheter or indwelling device may be coated with a gel-based formulation of the present invention. Such a coated device may be used in a method of treating or preventing a urinary tract infection in a patient when the device is placed in a patient.

Compositions containing one or more pharmaceutical excipients and one or more gel-based formulations may also be included in various types of gels, creams, or foams.

### BRIEF DESCRIPTION OF THE DRAWINGS

FIG. 1 is a line graph showing the inhibitory growth effects when a GML Gel (5% GML) formulation of the present invention is mixed with pathogenic microorganisms, and then allowed to grow when added to suitable media.

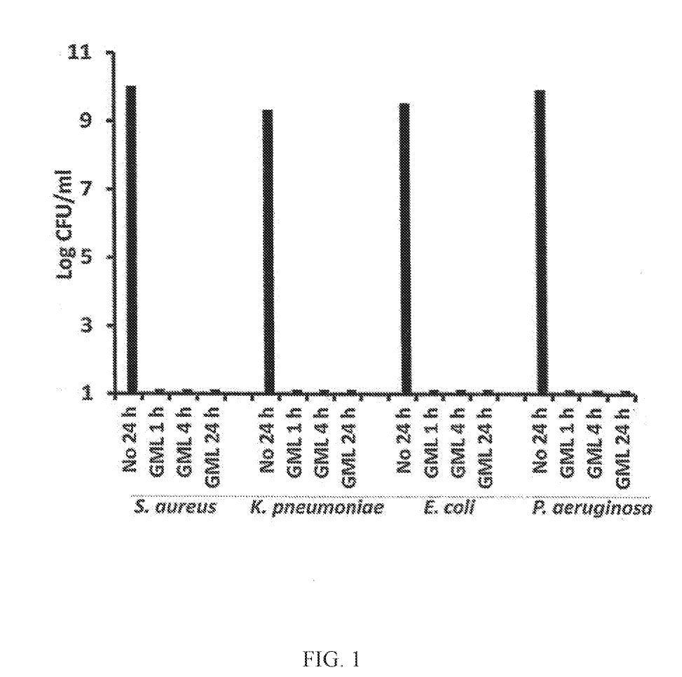

### DETAILED DESCRIPTION

The present invention provides topical GML gel-based formulations and methods of treating urinary tract infections with these formulations. In one embodiment, are used for treating urinary tract infections topically, for example, by facilitating delivery of effective amounts of GML or a GML-derivative to a skin or mucosal surface of a patient.

The term “antimicrobial” means effective in preventing, inhibiting, or arresting the growth or pathogenic effects of a microorganism. “Microorganism” is used herein to mean any bacteria, virus, or fungus. In one embodiment, the formulations of the invention are used to prevent, inhibit, or arrest the growth, for example, of one or more of the following microorganisms: S. aureus, P. aeruginosa, E. coli or K. pneumoniae.

The terms “antibacterial”, “anti-fungal”, or “anti-protozoan” refer to inhibition or arrest of the growth of a bacterium, fungus, or protozoans, or a reduction in the severity of or likelihood of developing a bacterial, fungal, or protozoan disease, inducing death of the bacterium, fungus, or protozoans, or reduction or inhibition of the pathogenic effects of the respective bacterium, fungus, or protozoans. “Bactericidal” is used interchangeably with “antibacterial.”

The term “anti-viral” refers to inhibition of viral infection or virus replication, a reduction in the likelihood that a patient exposed to a virus will contract the viral disease or a reduction in the severity of the viral disease.

The term “effective amount” refers to an amount that is sufficient to affect a beneficial or desired antimicrobial activity, including, without limitation, killing the microorganism or inhibiting microbial infection, growth or toxicity. An effective amount of GML is about up to 1 mg/mL, about up to 10 mg/mL, about up to 50 mg/mL, or about up to 100 mg/mL.

The terms “treat”, “treatment”, and “treating” refer to an approach for obtaining beneficial or desired results, for example, clinical results. For the purposes of this invention, beneficial or desired results may include inhibiting or suppressing the growth of a microorganism or killing a microorganism; inhibiting one or more processes through which a microorganism infects a cell or patient; inhibiting or ameliorating the disease or condition caused by a microbial infection; or a combination thereof. The terms “treat”, “treatment”, or “treating” also refer to prophylaxis treatment. “Prophylaxis” refers to prevention of an infection or disease, or prevention of the development of symptoms of that infection or disease, a delay in the onset of an infection or disease or its symptoms, or a decrease in the severity of a subsequently developed infection or disease or its symptoms.

The term “topical” refers to the application of the composition to any skin or mucosal surface. “Skin surface” refers to the protective outer covering of the body of a vertebrate, generally comprising a layer of epidermal cells and a layer of dermal cells. A “mucosal surface,” as used herein, refers to a tissue lining of an organ or body cavity that secretes mucous.

The term “pharmaceutically acceptable topical carrier” refers to a material, diluent, or vehicle that can be applied to skin or mucosal surfaces without undue toxicity, irritation, or allergic reaction.

The term “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “plant-derived oil” means a substance extracted from a plant or seed that exists in liquid form at room temperature. Suitable plant-derived oils include, without limitation, palm, olive, corn, canola, coconut, soybean, wheat germ, jojoba, sunflower, sesame, peanut, cottonseed, safflower, soybean, rapeseed, almond, beech nut, cashew, hazelnut, macadamia, mongongo nut, pecan, pine nut, pistachio, walnut, grapefruit seed, lemon, orange, bitter gourd, bottle gourd, buffalo gourd, butternut squash seed, egusi seed, pumpkin seed, watermelon seed, acai, black seed, blackcurrant seed, borange seed, evening primrose, flaxseed, eucalyptus, amaranth, apricot, apple seed, argan, avocado, babassu, coriander seed, grape seed, mustard, poppyseed, rice bran, castor, or mixtures thereof. Mixtures can be, by way of example and without limitation, a combination of olive oil and soybean oil, a combination of coconut oil and wheat germ oil, or a combination of jojoba oil, palm oil, and castor oil. Mixtures of suitable oils can be binary, ternary, quaternary, or higher mixtures.

The term “accelerant” refers to a compound, substance, liquid, powder, or mixture that, when added to GML or GML-derivative has the effect of enhancing or contributing to the antimicrobial properties of the composition.

The term “active ingredient” means an antibacterial ingredient, anti-fungal ingredient, anti-viral ingredient, anti-protozoan ingredient, or combination thereof. Antibacterials for use with the invention, for example, include aminoglycosides, carbacephems, cephalosporins, glycopeptides, lincosamides, lipopetides, macrolides, monobactams, nitrofurans, penicillins, polypetides, quinolones, sulfuramides, and tetracyclines. Anti-fungal ingredients include, without limitation, those of the azole class, polyene class, or echinocanins class, nucleoside analogues, allylamines, griseofulvin, tolnaftate, or selenium compounds. Anti-viral ingredients include, for example and without limitation, acyclovir, ganciclovir, valganciclovir, abacavir, enofovir, lamivudine, emtricitabine, zidovudine, tenofovir, efavirenz, raltegravir, enfuvirdide, maraviroc, ribavirin, amantadine, rimantadine, interferon, oseltamivir, and zanamivir.

The term “cellulose derivative” refers to any a cellulose-based compound and may include, for example, hydroxyethyl cellulose, hydroxypropyl cellulose, methylcellulose, ethylcellulose, hydroxypropyl methyl cellulose, or cellulose acetate.

The term “biofilm” means an aggregate of microorganisms, usually bacterial, adhered to one another and growing on a surface. The microbial cells in the biofilm typically produce an extracellular matrix known as an extracellular polymeric substance. Often, this matrix and the density of the aggregate itself significantly increase the antibiotic resistance of the bacteria in the biofilm. Biofilms can be involved in ear infections and dental diseases such as gingivitis.

The term “isolated compound” refers to a compound (e.g., GML or a related compound) that either has no naturally-occurring counterpart or has been separated or purified from components which naturally accompany it, e.g., in tissues such as pancreas, liver, spleen, ovary, testis, muscle, joint tissue, neural tissue, gastrointestinal tissue or tumor tissue, or body fluids such as blood, serum, or urine. Typically, a naturally occurring biological compound is considered “isolated” when it is at least 70%, by dry weight, free from other naturally-occurring organic molecules with which it is naturally associated. Preferably, a preparation of a compound for use in the invention is at least 80%, more preferably at least 90%, and most preferably at least 99%, by dry weight, that compound. The degree of isolation or purity can be measured by any appropriate method, e.g., column chromatography, polyacrylamide gel electrophoresis, or HPLC analysis. Since a compound (e.g., GML) that is chemically synthesized is, by its nature, separated from the components that naturally accompany it, the synthetic compound is by definition “isolated”. Isolated compounds, and supplementary ingredients useful for the invention, can be obtained, for example, by: (i) extraction from a natural source (e.g., from tissues or bodily fluids); (ii) where the compound or supplementary ingredients are proteins, by expression of recombinant nucleic acids encoding the proteins; or (iii) by standard chemical synthetic methods known to those in the art. In one embodiment, the composition provided herein comprises the monoglyceride GML. GML is a fatty acid ester of glycerol, derivative of lauric acid, with the chemical formula C₁₅H₃₀O₄. GML is also known in the art as glyceryl laurate or monolaurin. GML is found naturally in breast milk and some plants, and is used as a food and cosmetic additive. GML and other glycerides are listed in the Generally Recognized as Safe Substances database by the US Food and Drug Administration. GML and related compounds have been previously disclosed in U.S. Patent Publication No. 2007/0276049 (filed Nov. 10, 2004), U.S. Pat. No. 8,796,332, and U.S. Publication Number 2013/0281532.

GML can be obtained or synthesized in multiple forms including both R and S optical isomers, as well as forms with lauric acid in the 1/3-position and in the 2-position. The gel-based formulation provided herein, in one embodiment, comprises the R isomer of GML. In another embodiment, the formulation comprises the S isomer of GML. In yet another embodiment, the formulation comprises a racemic mixture of isomers.

Similarly, the formulation may comprise GML with lauric acid ester at the 1/3 position, GML with lauric acid ester at the 2-position, or a combination thereof. R and S isomers of each form and racemic mixtures thereof, are amenable for use with the present invention.

The chemical structure of GML with lauric acid in the 1 or 3 position is glycerol monolaurate (GML) 1/3-position.

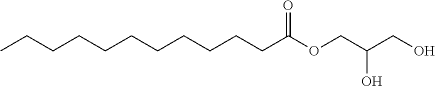 [SMILES: CCCCCCCCCCCC(=O)OCC(O)CO]

The chemical structure of GML with lauric acid ester in the 2-position is: Glycerol monolaurate (GML) 2-position.

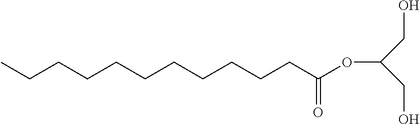 [SMILES: CCCCCCCCCCCC(=O)OC(CO)CO]

In another embodiment, the gel-based formulation comprises a GML derivative, for example a compound selected from one of Formulas A-F. Examples of such compounds include, by way of example and without limitation, glycerol monocaprylate, glycerol monocaprate, glycerol monomyristate, glycerol monopalmitate, and dodecyl glycerol.

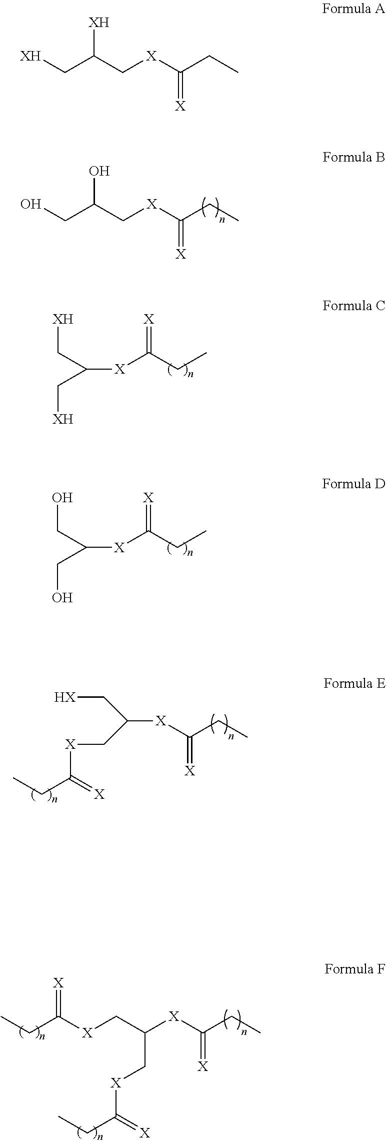

wherein each occurrence of X is independently —O— or —S—; and n is an integer from 5 to 20 (inclusive).

In another embodiment, the gel-based formulation comprises at least one derivative of GML, and the at least one derivative is a compound of either Formula E or Formula F. Examples of such compounds include, but are not limited to, glycerol dilaurate, glycerol dicaprylate, glycerol dimyristate, glycerol trilaurate, and glycerol tripalmitate.

In one embodiment, a compound of Formula A, B, C, or D is present in a formulation of the invention, and at least one —X— is —S—. In one embodiment, one occurrence of —X— is —S— and the remaining occurrences of —X— are —O—.

In one embodiment, a compound of Formula E or F is present in the formulation of the invention, each occurrence of n is 10, and at least one —X— is —O—.

The gel-based formulation provided herein, in one embodiment, comprises GML and a GML derivative. For example, in one embodiment, the gel-based formulation provided herein comprises GML and a compound of Formula F. In a further embodiment, each occurrence of n is 10 and at least one —X— is —O—.

In another embodiment, the gel-based formulation comprises GML or derivative thereof at a concentration of about 10 μg/mL to about 100 mg/mL. In a further embodiment, the gel-based formulation comprises GML or derivative thereof at a concentration of about 50 μg/mL to about 50 mg/mL. In a further embodiment, the gel-based formulation comprises GML or derivative thereof at a concentration of about 100 μg/mL to about 10 mg/mL. In yet a further embodiment, the gel-based formulation comprises GML or a derivative thereof at a concentration of about 500 μg/mL to about 5 mg/mL.

In one embodiment, the gel-based formulation comprises GML or derivative thereof at a concentration of about 10 μg/mL, about 50 μg/mL, about 100 μg/mL, about 500 μg/mL, about 1 mg/mL, about 5 mg/mL, about 10 mg/mL, about 50 mg/mL, or about 100 mg/mL.

Exemplary GML Concentrations

| | | |
|---|---|---|
| 0.001% | 10 | μg/mL |
| 0.01% | 100 | μg/mL |
| 0.1% | 1 | mg/mL |
| 1% | 10 | mg/mL |
| 2.5% | 25 | mg/mL |
| 5% | 50 | mg/mL |
| 7.5% | 75 | mg/mL |
| 10.0% | 100 | mg/mL |

The amount of GML or derivative thereof in the composition can be tailored accordingly to the extent of the urinary tract infection being treated as well as the characteristics of the patient being treated. The amount of GML in the composition may vary depending on, for example, the nature of the infection or illness; the site of administration; the patient's medical history, patient weight, age, sex, and surface area being treated; and whether the patient is receiving any other medications.

As provided above, in one aspect, the present invention is directed to a gel-based formulation comprising GML or a derivative thereof. In one embodiment, the gel-based formulation comprises at least one glycol. For example, in one embodiment, the gel-based formulation comprises propylene glycol, polyethylene glycol, or a combination thereof. In one embodiment, the polyethylene glycol has a molecular weight (MW) range from about 300 to about 10,000. In a further embodiment, the polyethylene glycol has a molecular weight of about 300 to about 1,000. In a still further embodiment, the polyethylene glycol has a molecular weight of about 400.

In one embodiment, polyethylene glycol is present in the gel-based formulation. In a further embodiment, the polyethylene glycol has a MW of about 400, about 500 or about 1,000. In one embodiment, the polyethylene glycol is present in the gel-based formulation at a concentration (w/w) of about 15% to about 50%, about 20% to about 40%, or about 25% to about 35%, for example, about 15%, about 20%, about 25%, about 30%, about 35%, about 40%, about 45%, or about 50%. In a further embodiment, both propylene glycol and polyethylene glycol are present in the gel-based formulation. In a further embodiment, propylene glycol is present at a concentration of about 70% to about 80% and polyethylene glycol is present at a concentration of about 20% to about 30%. In even a further embodiment, the polyethylene glycol is polyethylene glycol 400.

In a further embodiment, propylene glycol is present in the composition. In yet a further embodiment, propylene glycol is present in the composition at a concentration of about 60% to about 80%, for example, about 60%, about 65%, about 70%, about 71%, about 72%, about 73%, about 74%, about 75%, or about 80%.

In another embodiment, a gel-based formulation comprising GML or a derivative thereof is provided. In one embodiment, the gel-based formulation comprises at least one cellulose derivative. In a further embodiment, the composition comprises one cellulose derivative or two cellulose derivatives. In one embodiment, the cellulose derivative is hydroxypropyl cellulose. In another embodiment, the cellulose derivative is hydroxyethyl cellulose, carboxymethyl cellulose or hydroxymethyl cellulose. In yet another embodiment, the composition comprises a combination of hydroxyethyl cellulose and hydroxypropyl cellulose. In one embodiment, the cellulose derivative is present at a concentration of about 0.1% (w/w) to about 5.0% (w/w). In a further embodiment, multiple cellulose derivatives are present in the composition at the same concentration. In a further embodiment, two cellulose derivatives are present, and each is present at a concentration of about 1.25% (w/w). Cellulose derivatives include, for example, hydroxyethyl cellulose, hydroxypropyl cellulose, methylcellulose, ethylcellulose, hydroxypropyl methyl cellulose, or cellulose acetate.

In one embodiment, the gel-based formulation provided herein comprises GML or a derivative thereof, at least one cellulose derivative, propylene glycol and polyethylene glycol.

In another embodiment, a gel-based formulation comprising GML or a derivative thereof is provided. In a further embodiment, the composition comprises at least one plant-derived oil, for example, at least one of the oils described above (e.g., palm oil, olive oil, or corn oil). In one embodiment, the plant-derived oil is present in the composition at a concentration of as much as about 100 w/w %.

In one embodiment, the gel-based formulation provided herein comprises a plant-derived oil and at least one cellulose derivative. For example, in one embodiment, the gel-based formulation comprises hydroxypropyl cellulose and a plant-derived oil, or hydroxyethyl cellulose and a plant-derived oil, or a combination of hydroxypropyl cellulose, hydroxyethyl cellulose, and a plant-derived oil. In one embodiment, the cellulose derivative and the plant-derived oil (e.g., palm oil, corn oil, or plant oil), are each present at the same concentration (w/w). In another embodiment, the gel-based formulation comprises petroleum jelly. In one embodiment, the composition comprises a plant-derived oil and two cellulose derivatives. In a further embodiment, the two cellulose derivatives are hydroxypropyl cellulose and hydroxyethyl cellulose, and the total concentration of cellulose derivatives in the composition is about 1.25% (w/w). Cellulose derivatives include, for example, hydroxyethyl cellulose, hydroxypropyl cellulose, methyl cellulose, ethyl cellulose, hydroxypropyl methyl cellulose, or cellulose acetate.

In some embodiments, the gel-based formulation provided herein comprises one or more accelerants. In a further embodiment, the accelerant is an organic acid, a chelator, or a combination thereof. In a further embodiment, the accelerant is a chelator. In even a further embodiment, the accelerant is EDTA.

The accelerant, in one embodiment, is EDTA. In a further embodiment, the GML composition provided herein comprises EDTA at a concentration of about 0.00005 M, about 0.0005 M, about 0.005 or about 0.05 M. In another embodiment, a chelator is present in the composition at a concentration of about 0.00005 M to about 0.05 M, about 0.0005 M to about 0.005 M, or about 0.005 to about 0.05 M.

In one embodiment, the gel-based formulation comprises both a plant-derived oil and an accelerant, for example palm oil and EDTA. In another embodiment, the accelerant is an organic acid and is present in the formulation with a plant-derived oil. In one embodiment, the gel-based formulation provided herein comprises an accelerant and a non-aqueous gel, for example a gel comprising a cellulose derivative. In another embodiment, the gel-based formulation comprises GML or a derivative thereof, a plant-derived oil, a non-aqueous gel (e.g., a gel comprising one or more cellulose derivatives) and an accelerant.

In one embodiment, the composition contains at least one pharmaceutically acceptable excipient. Pharmaceutically acceptable excipients are well known to those skilled in the art and may include buffers (e.g., phosphate buffer and citrate buffer), amino acids, alcohols, proteins such as serum albumin, parabens (e.g., methylparaben), or mannitol.

In one embodiment, the pH of the composition is from about 3.5 to about 7.0. In a further embodiment, the pH of the composition is from about 4.0 to about 6.0. In a still further embodiment, the pH of the composition is from about 4.0 to about 4.5.

In one embodiment, the composition provided herein comprises GML or a derivative thereof and a pharmaceutically acceptable topical carrier. In one embodiment, the pharmaceutically acceptable topical carrier is a mix of hydrocarbons such as, for example, paraffin wax or petroleum jelly. Petroleum jelly is any water-insoluble, hydrophobic, semi-solid mixture of hydrocarbons. The pharmaceutically acceptable topical carrier can be added to any of the formulations described herein.

In one embodiment the gel-based formulation comprises an additional active ingredient. Additional active ingredients include, for example, antibacterial, anti-viral, anti-fungal, and anti-protozoan ingredients. Antibacterials include, without limitation, aminoglycosides, carbacephems, cephalosporins, glycopeptides, lincosamides, lipopetides, macrolides, monobactams, nitrofurans, penicillins, polypetides, quinolones, sulfuramides, or tetracyclines. Anti-fungal ingredients include, without limitation, those of the azole class, polyene class, or echinocanins class, nucleoside analogues, allylamines, griseofulvin, tolnaftate, or selenium compounds. Anti-viral ingredients include, for example and without limitation, acyclovir, ganciclovir, valganciclovir, abacavir, enofovir, lamivudine, emtricitabine, zidovudine, tenofovir, efavirenz, raltegravir, enfuvirdide, maraviroc, ribavirin, amantadine, rimantadine, interferon, oseltamivir, or zanamivir.

In another embodiment, the composition is a solid, semi-solid, foam, wax, cream, or lotion.

The GML gel-based formulations described herein may be less irritating than currently approved antimicrobial compositions, therefore resulting in a more favorable patient compliance rate, as compared to other antimicrobial compositions presently used in the art.

In one embodiment, the method comprises administering to the patient a gel-based formulation comprising GML or a derivative thereof, as described herein. In one embodiment, the method comprises topically administering to the patient an effective amount of a composition comprising GML or a derivative thereof, a plant-derived oil, and a pharmaceutically acceptable topical carrier. In another embodiment, the method comprises topically administering an effective amount of a composition comprising GML, a non-aqueous gel, and a pharmaceutically acceptable topical carrier. For example, the composition may be given twice per day for 3-4 days, or 6-7 days. Alternatively, the composition may be given once per day for 7-10 days or 12-14 days.

In one embodiment, the method of treating a microbial infection comprises applying an effective amount of one or more of the GML compositions described herein to at least one skin or mucosal surface of a patient.

In some embodiments, the gel-based formulation is applied to or impregnated in a wipe, sponge, swab, or other material, and then applied to the skin or mucosal surface of the patient using the respective material. As used herein, the term “swab” refers to a material suitable for applying a liquid, gel, wax, cream, or lotion to a skin or mucosal surface, or the act of applying a liquid, gel, wax, cream, or lotion to the skin or mucosal surface, or the act of collecting a liquid, gel, wax, cream, lotion, or fluid from the skin or mucosal surface. In some embodiments, the material is attached to a holder, for example a stick, wire, rod, or applicator. In further embodiments, the material attached to a holder is attached at one or both ends thereof. In some embodiments, the wipe, sponge, swab, or other material is pre-loaded or packaged together with the composition.

In other embodiments, the gel-based formulation is applied to or impregnated in a urinary catheter or other indwelling device ant the coated device is then placed in a patient using known processes and procedures.

GML compositions inhibit microbial infection through one or more of several mechanisms that include, but are not limited to, direct microbial toxicity; inhibiting entry of the infectious microorganism into the vertebrate cell; inhibiting growth of the microorganism; inhibiting production or activity of virulence factors such as toxins; stabilizing the vertebrate cells; or inhibiting induction of inflammatory or immunostimulatory mediators that otherwise enhance the infectious process.

In one embodiment, direct GML-mediated interruption of bacterial membranes includes interference with the localization of signaling proteins within the membrane, or interference with ligand binding to signaling proteins. In one embodiment, GML has an indirect effect on a two-component signal transduction system and the effect is selected from modifications to membrane structure that interfere with the ability of transmembrane proteins to perform signaling functions; dissipation of the bacterial plasma membrane potential; and alterations of pH gradients across the membranes.

Similar to GML's putative effects on bacterial plasma membranes, GML has been shown to inactivate certain viruses by disrupting viral lipid envelopes.

Methods of identifying and diagnosing a bacterial, viral, fungal, or protozoan infection are generally known by those skilled in the art. To assess whether the formulations disclosed herein are useful to treat an infection, methods known to those of ordinary skill in the art may be employed.

In one embodiment, a method is provided to remove or kill a biofilm comprising one or more microorganisms. In one embodiment, the method comprises administering the gel-based formulation by applying it directly to the biofilm. In some embodiments, the methods of the invention comprise administering a second active ingredient, along with GML or a derivative of GML. The additional active ingredient may be present in the compositions described herein, or may be administered separately. In one embodiment, the one or more additional active ingredients prior to, or after, the topical GML composition is administered. For example, the two active ingredients may be topically administered serially, or administered serially by different routes of administration.

In one embodiment, the additional active ingredient (s) is administered before, during, or after administration of the composition of the invention. In another embodiment, the additional active ingredient(s) is administered by the same route as the composition or by a different route. For example, the additional active ingredient(s), in one embodiment, is administered by one of the following routes of administration: topical, intranasal, intradermal, intravenous, intramuscular, oral and subcutaneous. The dose of additional active ingredients depends on, for example, the nature of the infection or illness; the site of administration; patient weight, age, sex, and surface area; concomitant medications; and medical judgment.

### EXAMPLES

The present invention is further illustrated by reference to the following Examples. However, it should be noted that these Examples, like the embodiments described above, are illustrative and are not to be construed as restricting the scope of the invention in any way.

### Example 1

5% w/v GML nonaqueous gel is bactericidal for 54 strains of S. aureus, including highly antibiotic resistant organisms and multiple clonal groups. GML is antimicrobial on contact, killing the organisms in only a few minutes. The estimated chance of S. aureus developing resistance to GML is <1/10; thus resistance is highly unlikely. 5% GML nonaqueous gel is also a stronger anti-staphylococcal ingredient than GML alone.

### Example 2

To directly assess the effect of GML on the formation of biofilms, 96 well plastic microtiter plates were inoculated with approximately 10⁶/mL of one of three strains of S. aureus (MN8, a methicillin sensitive strain; MNWH, a methicillin resistant strain; or MW2, a methicillin resistant strain), or with non-typable Haemophilus influenzae. Wells were cultured stationary at 37° C. for 24 and 48. As a control, in one set of three wells for each microbe, the wells were agitated 3 times by pipetting up and down. The bactericidal activity of GML was determined by measuring CFU/mL in supernatants. After removal of supernatants, wells were washed three times with PBS to remove unbound cells, and were then treated with crystal violet for 30 minutes. Wells were again washed three times with PBS to remove unbound crystal violet. Finally, wells were treated with ethanol to solubilize biofilm associated crystal violet. Absorbance at 595 nm was determined by an ELISA reader to measure biofilm formation.

Growth of all three S. aureus strains was completely inhibited by GML at 500 μg/mL at both 24 and 48 hours, as measured by CFU/mL. In contrast, at 10 fold lower GML concentrations than necessary to inhibit bacterial growth, biofilm formation was significantly inhibited as measured by reduced crystal violet staining of retained biofilm material in wells of the microtiter plates.

### Example 3

5% GML nonaqueous gel, GML with other accelerants, such as low pH and EDTA, is bactericidal to Pseudomonas species on contact. Bactericidal is defined as a greater than three log reduction in bacterial colony-forming units per milliliter, compared to starting inoculum.

Modifications and variation of the above-described embodiments of the invention are possible without departing from the invention, as appreciated by those skilled in the art in light of the above teachings. It is therefore understood that, within the scope of the claims and equivalents, the invention may be practiced otherwise than as specifically described.

1-22. (canceled)

23. A gel-based formulation to treat or prevent urinary tract infection comprising a composition that kills, or inhibits the growth of, one or more pathogenic microorganisms that cause urinary tract infections, where the composition comprises about 0.0001-0.05 M of an accelerant and about 10-100 mg/mL of an active compound selected from the group consisting of Formula 1, Formula 2, and a combination of Formulas 1 and 2, 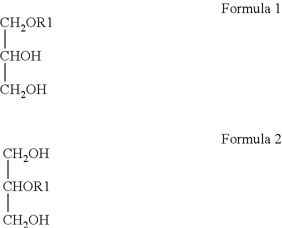
wherein R1 is: CO(CH₂)₁₀CH₃.

24. The gel-based formulation of claim 23, wherein the accelerant comprises lactic acid, ascorbic acid, citric acid, ethylenediaminetetraacetic acid, other chelating ingredients, or combinations thereof.

25. The gel-based formulation of claim 23, further comprising a plant-derived oil.

26. The gel-based formulation of claim 23, further comprising a cellulose derivative selected from the group consisting of hydroxyethyl and hydroxypropyl cellulose

27. The gel-based formulation of claim 23, further comprising a glycol derivative selected from the group consisting of polyethylene and propylene glycol.

28. The gel-based formulation of claim 23, further comprising petroleum jelly or a derivative thereof.

29. The gel-based formulation of claim 23, further comprising water and/or saline.

30. The gel-based formulation of claim 23, further comprising an additional active ingredient selected from the group consisting of an antibacterial, an anti-viral, an anti-fungal, an anti-protozoan, and combinations thereof.

31. The gel-based formulation of claim 23, wherein the gel-based formulation has a pH of about 4-4.5

32. A gel-based formulation of claim 23, wherein the gel-based formulation comprises about 0.0001-0.05 M of ethylenediaminetetraacetic acid and a topical solution comprising about a) 73.55 w/w % propylene glycol; b) 25 w/w % polyethylene glycol 400; c) 1.25 w/w % hydroxyethyl cellulose or hydroxypropyl cellulose; and d) saline and/or water.

33. A gel-based formulation of claim 23, wherein the gel-based formulation comprises 0.0001-0.05 M of ethylenediaminetetraacetic acid, 10-100 mg/mL glycerol monolaurate, and a topical solution comprising plant-derived oil; petroleum jelly, or derivative thereof, wherein the plant-derived oil is selected from the group consisting of palm oil, olive oil, corn oil, and combinations thereof, and the gel-based formulation has a pH of 4-4.5.

34. A urinary catheter or indwelling device coated with a gel-based formulation of claim 23.

35. A urinary catheter or indwelling device of claim 34, wherein the gel-based formulation further comprises an anti-biotic, an anti-viral, an anti-fungal, an anti-protozoan, or a combination thereof.

36. The use of a urinary catheter or indwelling device to treat or prevent a urinary tract infection in a patient comprising coating the urinary catheter or indwelling device with a gel-based formulation of claim 23.

37. A method of treatment or prophylaxis, the method comprising:
(a) identifying a patient having a urinary tract infection or at risk of such an infection that is or could be caused by one or more pathogenic microorganisms; and
(b) administering to the patient a gel-based formulation that (i) kills, or inhibits the growth of, the one or more pathogenic microorganisms, and (ii) comprises about 0.0001-0.05 M of an accelerant and about 10-100 mg/mL of an active compound selected from the group consisting of Formula 1, Formula 2, and a combination of Formulas 1 and 2: 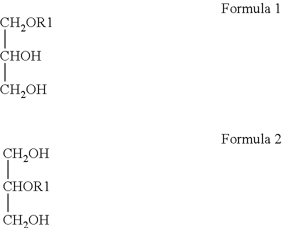
wherein R1 is: CO(CH₂)₁₀CH₃.

38. The method of claim 37, wherein the accelerant comprises lactic acid, ascorbic acid, citric acid, ethylenediaminetetraacetic acid, other chelating ingredients, or combinations thereof.

39. The method of claim 37, wherein the gel-based formulation further comprises a plant-derived oil, petroleum jelly or a derivative thereof.

40. The method of claim 37, wherein the gel-based formulation further comprises a gel of derivative selected from the group consisting of hydroxyethyl and hydroxypropyl cellulose, and a glycol derivative selected from the group consisting of polyethylene and propylene glycol.

41. The method of claim 37, wherein the gel-based formulation further comprises an additional active ingredient selected from the group consisting of an antibacterial, an anti-viral, an anti-fungal, an anti-protozoan, and combinations thereof.

42. The method of claim 37, wherein the gel-based formulation has a pH of about 4-4.5
